(19) United States
(12) Patent Application Publication
(10) Pub. No.: US 2008/0206204 A1
(43) Pub. Date: Aug. 28, 2008

(54) Human parthenogenetic stem cells

(76) Inventors: Tiziana Brevini, Segrate (IT); Fulvio Gandolfi, Segrate (IT); Guido Ragni, Milano (IT)

Correspondence Address: ARENT FOX LLP, 1050 CONNECTICUT AVENUE, N.W., SUITE 400, WASHINGTON, DC, 20036, US

(21) Appl. No.: 11/711,152

(22) Filed: Feb. 27, 2007

(51) Int. Cl. C12N 5/08

(52) U.S. Cl. 424/93.7; 435/366

(57) ABSTRACT

The invention provides a method for establishing pluripotent cell lines from human parthenotes, the uses of said cell lines for producing differentiated cells or tissues and for therapeutic applications especially in regenerative medicine.

The present invention provides methods for the establishment of cell lines from human parthenotes and the use thereof for making differentiated cells and tissues.

### BACKGROUND OF THE INVENTION

Parthenogenesis is the process of embryonic development without the intervention of a spermatozoon that, in lower species, can lead to the generation of new individuals. The embryos obtained by parthenogenesis (parthenotes) can develop to different stages after oocyte activation, depending on the species. Mammalian parthenotes are inherently unable to develop to term but can reach the blastocyst stage when an inner cell mass, the source of embryonic stem cells (ESCs), is formed. Parthenogenetic stem cells have been derived in mouse¹ and in non-human primates² but, so far, it has not been possible to obtain cell lines from human parthenotes³.

The use of human embryos to derive embryonic stem cells (ES cells) is ethically controversial and in some countries a ban exists on the use of these cells for research purposes. It is generally accepted, however, that ES cells have numerous beneficial applications, especially in the regeneration or replacement of damaged cells or tissues. ES cell-based treatments may include, for example, replacing destroyed dopamine-secreting neurons in a Parkinson's patient's brain, transplanting insulin-producing pancreatic beta cells in diabetic patients and infusing cardiac muscle cells in a heart damaged by myocardial infarction, as well as many other applications especially in regenerative medicine.

The importance of generating pluripotent lines of stem cells from sources other than “normal” human embryos (i.e. those generated by fertilized oocytes) is therefore immediately evident.

### DISCLOSURE OF THE INVENTION

In a first aspect, the invention provides a method in vitro for establishing pluripotent cell lines (or parthenogenetic stem cells) from human parthenotes, which comprises:
a) exposing a non-inseminated human oocyte to ionomycin and 6-dimethylaminopurine (6-DMAP), to induce its parthenogenetic activation;
b) maintaining the activated oocyte in culture conditions permissive for its development to blastocyst;
c) isolating the inner cell mass (ICM) and plating the ICM cells on a STO fibroblasts feeder layer, whereby cell colonies are formed;
d) separately propagating the cell colonies on mitotically inactivated fibroblast feeder layers, whereby cell lines are established;
e) selecting the cell lines expressing the following human stem cell markers: octamer-binding transcription factor 4 (Oct-4), Nanog, Reduced expression protein-1 (Rex-1), alkaline phosphatase, Stage Specific Embryonic Antigen 4 (SSEA-4), Tumor Rejection Antigen TRA 1-81.

In a preferred embodiment, the oocyte activation under step a) is carried out by exposing the oocytes to 5 μM ionomycin followed by incubation with 2 mM 6-DMAP at 37° C.

In a further preferred embodiment, step c) comprises removing the zonae pellucidae by incubating blastocysts in the presence of pronase and plating the ICM cells on STO fibroblast feeder layers freshly inactivated with mitomycin-C.

In a yet further preferred embodiment, according to step d) the cell colonies are propagated on STO feeder layer in low glucose DMEM/F10 (1:1) medium supplemented with LIF and basic FGF.

The selected parthenogenetic stem cells are morphologically characterized by a small cytoplasmic/nuclear ratio, numerous nucleoli and form well defined colonies. These cell lines can be propagated extensively in vitro and constantly express the cell markers that characterize human stem cells. When cultured under appropriate conditions, the cell lines of the invention are able to differentiate generating a large variety of specialized cell types, including muscle cells, cardiomyocytes, adipocytes, epithelial cells, hepatocytes, cartilage cells, particularly neurons and hematopoietic cells. Advantageously, differentiated human cell types derived in vitro by parthenogenesis eliminate the necessity of producing or disaggregating normal, competent embryos. The procedures used for differentiating human embryonic stem cells can be equally applied to the differentiation of parthenogenetic stem cells. For a review on this subject, see Hoffman L, Carpenter M K “Characterization and culture of human embryonic stem cells” Nat Biotechnol 2005, 23: 699-708; Priddle H, Jones D R, Burridge P W, Patient R “Hematopoiesis from human embryonic stem cells: overcoming the immune barrier in stem cell therapies” Stem Cells 2006, 24: 815-824; Sonntag K C, Sanchez-Pernaute R “Tailoring human embryonic stem cells for neurodegenerative disease therapy” Curr Opin Investig Drugs 2006, 7: 614-618, which are herein incorporated by reference.

In a further aspect the invention relates to the use of human pluripotent cells, as herein provided, for in vitro generation of differentiated cells, preferably neurons, muscle cells, cardiomyocytes, adipocytes, epithelial, liver, cartilage and hematopoietic cells. The differentiated human cells in turn can be used in a variety of therapeutic applications, including, but not limited to, the treatment of neurological and neurodegenerative disorders, such as Parkinson's and Alzheimer's diseases, spinal cord injuries, muscular distrophy, heart diseases, graft-vs-host disease, diabetes, multiple sclerosis, vascular diseases, hypercholesterolemia, age-related disturbances, ulcers, burns, rejection of transplanted organs or tissues. In addition, the parthenogenetic cell-lines can be genetically modified and used for delivering therapeutic genes to individuals affected by genetic disorders, such as cystic fibrosis, breast cancer, hemophilia, Huntington's disease, muscular dystrophy, schizophrenia, sickle cell anemia, spine bifida, etc.

The parthenogenetic cell lines according to the invention can be cryo-conserved and, at the moment of use, thawed and propagated in culture in the presence of appropriate stimuli to induce differentiation.

Since the parthenotes contain a double set of oocyte's chromosomes, they carry half the normal combination of antigens, making them more compatible with the genetic and immunologic profile of recipient patients.

### DESCRIPTION OF THE FIGURES

FIG. 1—Characterization of human parthenogenetic cell lines. Expression of pluripotency markers:(ABC) Expression of OCT4; (DEF) Expression of SSEA4; (GHI) Expression of Alkaline Phosphatase; (LMN) Expression of TRA 1-81.

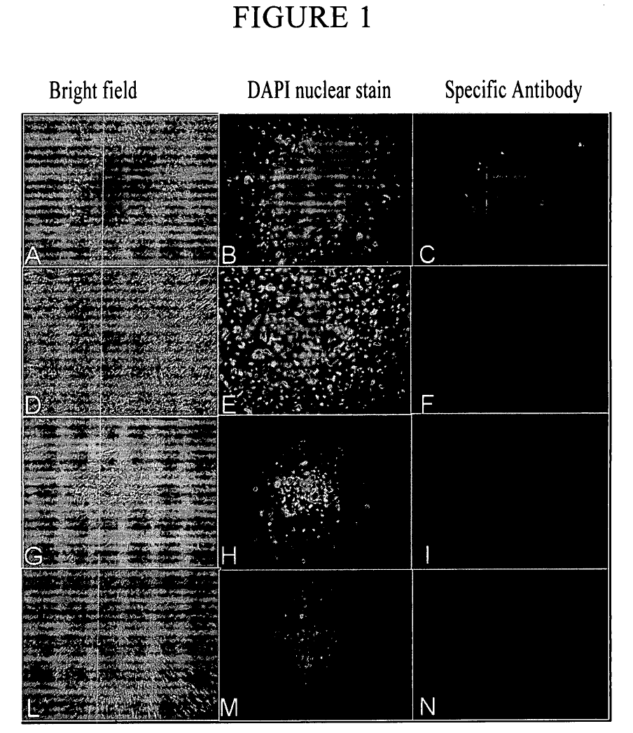

FIG. 2—Characterization of human parthenogenetic cell lines. Expression of telomerase activity. HP1 and HP3 cells, maintained in the undifferentiated state on mouse feeder layers, express telomerase activity that diminishes to undetectable levels upon differentiation and heat inactivation.

FIG. 3—Characterization of human parthenogenetic cell lines. Expression of CD45 and CD34 surface markers. Cells were separated by flow cytometry upon differentiation treatment

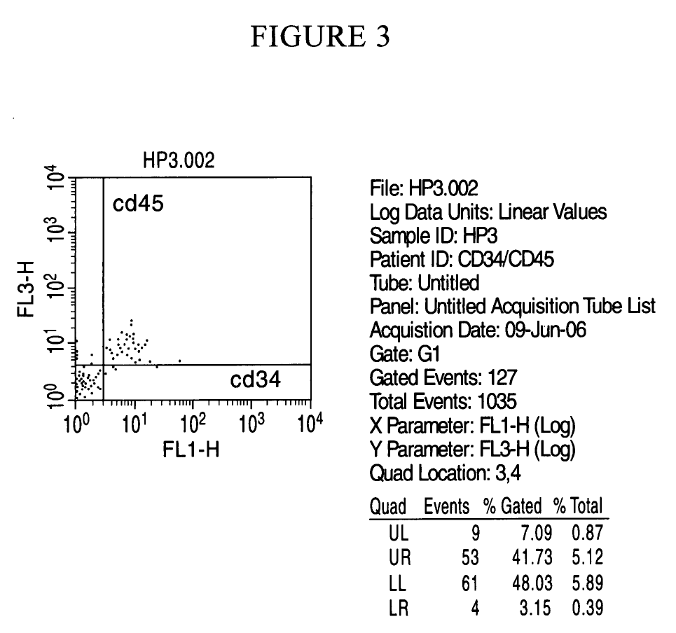

FIG. 4—Characterization of human parthenogenetic cell lines. HP cells show distinct hematopoietic cell morphology upon extensive differentiation in culture.

FIG. 5—Characterization of human parthenogenetic cell lines. Expression of neural differentiation markers: A) Expression of Nestin; B) Expression of MAP2; C) Expression of CNPase.

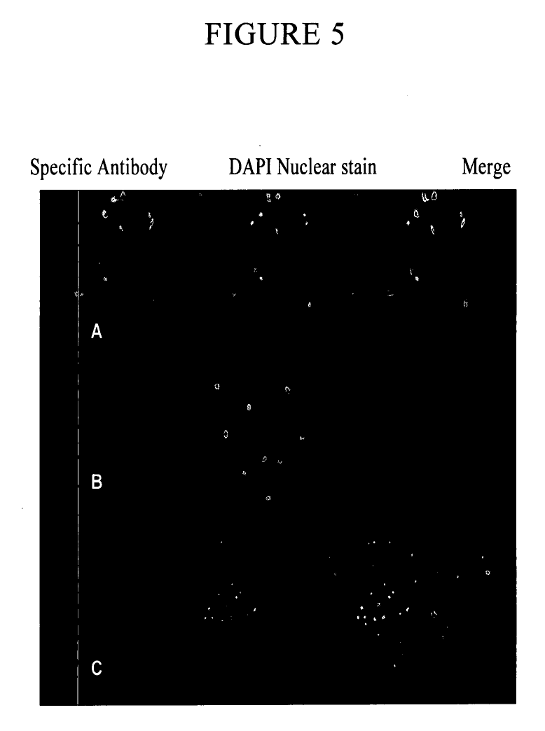

### EXPERIMENTAL PROCEDURES

Human Oocytes

Ooocytes were recovered from 4 patients with average age of 35,75 years (range 32-39). Activation was performed on 20 oocytes and 5 blastocysts were obtained.

Isolation and Plating of ICMs From Human Parthenogenetic Embryos

Zonae pellucidae were removed incubating blastocysts through two 50 μl droplets of pronase 0.5% w/v in medium TCM199 supplemented with 6.5 mg/ml HEPES, 1.1 mg/ml sodium bicarbonate, 4 mg/ml bovine serum albumin (BSA), 75 μg/ml penicillin and 50 μg/ml streptomycin. Blastocysts were left in the second drop for 7 minutes under mineral oil. Progression of zona digestion was carefully monitored under a stereo-microscope and was carried out at 38.5° C. on a thermostatically controlled stage. Blastocysts were then delicately washed through three drops of the medium described above but without pronase and supplemented with 20% (v/v) fetal bovine serum (FBS) (Gibco, Invitrogen Italy). They were then washed through three drops of HP medium (see below). In the third drop inner cell masses (ICM) were microsurgically removed. They were quickly moved through two drops of HP medium without oil overlay and singly plated on freshly inactivated STO fibroblast feeder layers in 4-well culture dishes (Nunc, DK).

Preparation of STO Fibroblast Feeder Layers

STO fibroblasts (LGC Promochem-ATCC, Italy) were routinely cultured in high glucose DMEM, supplemented with 2 mM glutamine, 75 μg/ml penicillin, 50 μg/ml streptomycin and 10% FBS. Fibroblasts were spilt every four days, just before reaching confluence.

For growth inactivation, sub-confluent mono-layers were exposed to the medium above containing 10 μg/ml mitomycin-C for 3 hours. They were washed four times with PBS and subsequently removed with 0.25% trypsin −0.02% EDTA. They were re-suspended in culture medium and seeded at a density of 25×10⁴ cell/well in 4-well dishes (Nunc, DK) coated with 0.1% gelatin. Inactivation was carried-out 24 hours before plating of ICMs or passaging of embryonic cells. Two hours before use, the medium was changed and replaced with HP medium.

Establishment and Culture of Cell Lines from Human Parthenogenetic Embryos

Cells were cultured in 5% CO2 at 37° C. in HP medium that consisted of low glucose DMEM/F10 (1:1) medium, supplemented with 2 mM glutamine, 1000 IU/ml of mouse recombinant LIF (ESGRO Gibco), 5 ng/ml human recombinant basic Fibroblast Growth Factor (R&D System, USA), 0.1 mM 2-mercaptoethanol, 75 μg/ml penicillin and 50 μg/ml streptomycin, 1% non-essential amino acids (Gibco, Invitrogen Italy), 1% nucleoside mix, 10% Knock-out serum replacer (Gibco, Invitrogen Italy) and 5% FBS. (Gibco, Invitrogen Italy). Within 3 days, circular colonies with distinct margins of small, round cells were observed (HP cells). When a colony enlarged enough to cover half or more of the well surface, cells were mechanically removed using a sterile microloop (Nunc, DK), they were transferred to a 50 μl drop of fresh medium and pipetted to small cell clumps, avoiding to obtain single cell suspension. Cells were then passaged on freshly prepared feeder-layers. Culture medium was changed every day.

Gene Expression of the Human Parthenogenetic Cell Cultures

The expression of a gene panel (as indicated in table 1) was examined by RT-PCR on part of the cells at each passage. RNA was isolated using the acid-phenol method according to Chomczynski and Sacchi¹; in addition, a small amounts of poly(A) RNA (4 ng/μl) was added as carrier to the lysate before homogenization. RNA extraction procedure included a DNase I (1 U/μl, Invitrogen, Italy) incubation of 15′ min at room temperature, then 25 mM EDTA was added to the sample in order to inactivate the enzyme for 10 min at 65° C.

RNA was then immediately used for reverse transcription (RT), which was carried out in a total volume of 20 μl of reaction mixture consisting of 8.5 μl of sterile water, 1 μl of 10 mM dNTPs and 1 μl of oligo(dT)12-18-primer. RNA was denatured at 65° C. for 5 min, then 4 μl of 5× First-Strand Buffer (250 mM Tris-HCl pH 8.3, 375 mM KCl, 15 mM MgCl₂), 1.5 μl MgCl₂ 50 mM, 2 μl of 0.1 M DTT and 1 μl of RNaseOUT-TM Recombinant Ribonuclease Inhibitor (40 U/μl) were added. Reverse transcription was performed with 200 U (1 μl) Superscript-TM II Reverse Transcriptase for 1 hr at 42° C. Enzymes were inactivated at 70° C. for 15 min. Tubes without RNA or reverse transcriptase were prepared as negative controls.

Polymerase Chain Reaction (PCR) was performed in a final volume of 20 μl, 0.3 μl of Taq DNA Polymerase (5 U/μl), 2 μl of 10× PCR Buffer (200 mM Tris-HCl pH 8.4, 500 mM KCl), 1 μl of 10 mM dNTPs, 1 μl (100 pmol) of each sequence-specific primer. Amplifications were carried out in an automated thermal cycler (iCycler, Biorad), using the conditions appropriate for each set of primers. PCR primers were designed using the oligo program Primer3 Input. The accession numbers, the sequences of the primers used, the sequence references (when available) the annealing temperatures and the fragment sizes have been summarized in Table 1.

RT-PCR products were subjected to electrophoresis on a 2% agarose gel in 1× TAE buffer (40 mM Tris-acetate, 1 mM EDTA) containing 0.5 μg/ml ethidium bromide (EtBr). The same EtBr concentration was present in the running buffer. After electrophoresis at 80V for 45 min, the fragments were visualized on a 312 nm UV trans-illuminator. The image of each gel was recorded using a digital Kodak camera (DC290). Amplification products were purified in Spin-X centrifuge tube filters (Corning, the Netherlands), sequenced (SEQLAB, Gottingen, Germany) and aligned using Clustal W 1.82 (EMBL-EBI service).

Derivation of Embryoid Bodies

To induce the formation of embryoid bodies (EBs), HP cells were cultured in 30 μl hanging droplets as previously described of HP medium, without LIF and bFGF. The medium was refreshed every day and after 7-9 days, well formed cavitating EBs were detectable. Differentiation of EBs was confirmed, through both morphological examination and molecular analysis that demonstrated the expression of markers (see Table 1) related to mesoderm (Bone Morphogenetic Protein-4, BMP-4), ectoderm (Neurofilament-H, NF-H), endoderm (alpha-amilase) and trofectoderm (interferon-t IFN-τ). All these markers were expressed by day 9 of culture.

Telomerase Activity Detection

Telomerase activity is correlated with immortality and is typically present in germ cells, cancer cells and embryonic stem cells (Kim et al., 1994; Armstrong et al., 2000; Amit et al., 2000).

Telomerase activity was measured by using the TRAPeze Kit (Chemicon) as recommended by the manufacturer. The TRAP assay is a primer extension assay in which telomerase synthesizes telomeric repeats onto oligonucleotide primers. The telomerase extension products serve as a template for PCR amplification. The laddering in the polyacrylamide gel represents increasing numbers of telomeric repeats. Control template, buffer and control extract were supplied by the TRAPeze kit. Extract from undifferentiated and differentiated HP cells were normalized to the protein concentration. Heat inactivated extracts, which were included in every experiments, were boiled for 10 minutes before the assay.

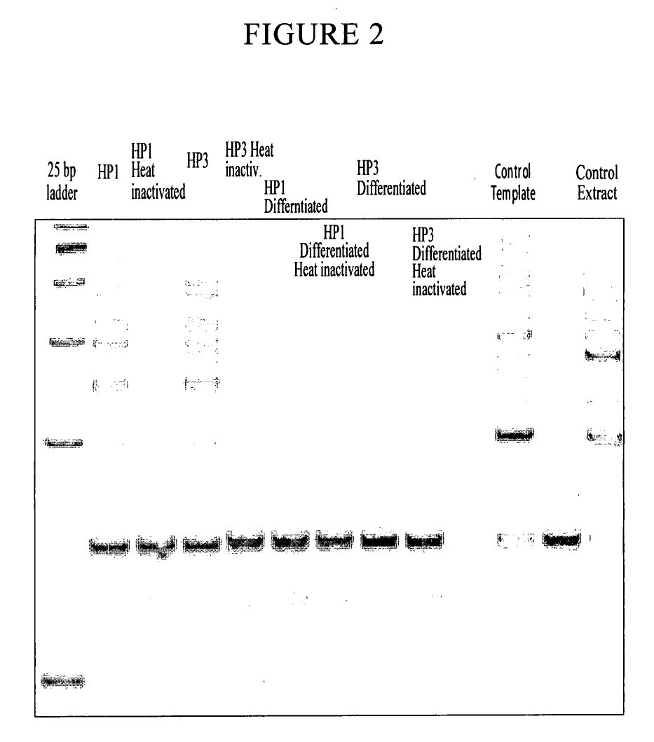

As expected, undifferentiated HP cells displayed telomerase activity; upon differentiation telomerase activity disappeared with no signal detectable in fully differentiated cells. These data indicate that HP cells exert a control on telomerase activity which is comparable to the one described for bi-parental stem cells (Armstrong et al., 2000; Amit et al., 2000).(FIG. 2)

Spontaneous Differentiation of HP Cell Lines

Embryoid bodies were mechanically dissociated and cells were plated directly on CultureWell Chambered Coverglass 16-well dishes (Molecular Probes Europe, the Netherlands) to encourage adherent culture conditions and spontaneous differentiation in HP medium without LIF, bFGF and Knock-out serum replacer but supplemented with 15% FBS. After one week the medium was removed, cells were rinsed in PBS, fixed in 4% para-formaldehyde and permeabilized with 0.1% Triton X100. Cells were then stained with antibodies specific for human vimentin, desmin, keratin-17, and β-tubulin III (Chemicon, USA). Secondary detection was carried out with Alexa Fluor antibodies (Invitrogen, Italy). The results obtained were observed under a TCS-NT laser confocal microscope (Leica Microsystems, Germany). Upon differentiation all the above mentioned proteins were expressed in cells derived from the undifferentiated cell lines.

Neural Differentiation

Embryoid Bodies were prepared in Human Parthenogenetic Cell medium without LIF and bFGF, with 15% Knock-out serum replacer. 5% FBS and supplemented with 10 μM retinoic acid and 10 ng/ml Sonic Hedgehog (R&D System, USA).

They were kept 48 hours in hanging drops culture condition, with the medium refreshed every day and then they were dissociated and plated on 0.1% gelatin coated CultureWell Chambered Coverglass 16-well dishes (Molecular Probes Europe, the Netherlands). Differentiation was carried out in Neural Progenitor Cell Basal Medium (Cambrex Bioscience, USA), supplemented with Neural Progenitor Growth Singlequots (Cambrex Bioscience, USA) and 25 ng/ml Brain Derived Neurotrophic Factor (R&D System, USA). After a period from 9 to 21 days of culture, cells were fixed and stained with specific antibodies for: nestin, microtubule associated protein-2 (MAP-2), neuron specific beta III tubulin, 2′,3′-cyclic nucleotide 3′-phosphodiesterase (CNPase) (Abcam, UK). The results obtained were observed under a TCSNT laser confocal microscope (Leica Microsystems, Germany). After a period from 9 to 21 days of culture, cells were fixed and stained with antibodies specific for neural differentiation marker molecules. The results obtained were observed under a TCS-NT laser confocal microscope (Leica Microsystems, Germany). Cells demonstrated positive staining for the early neural differentiation marker nestin after 9 days of culture and displayed positivity for markers related to more mature forms of neural differentiation like neuron specific beta III tubulin, microtubule associated protein-2 (MAP-2) and 2′,3′-cyclic nucleotide 3′-phosphodiesterase (CNPase) (Abcam, UK) when differentiation was extended to longer culture periods.

Hematopoietic Differentiation

Embryoid Bodies were prepared in Human Parthenogenetic Cell medium without LIF and bFGF, with 15% Knock-out serum replacer. 5% FBS. They were cultured in hanging drops culture condition, with the medium refreshed every day. After 9 days EBs were disaggregated into small clumps of cells and re-suspended in culture for two weeks in medium containing cytokines and 10% FBS.

Samples of cells were then subjected to flow-cytometry analysis for CD45 and CD34 in order to check progression of differentiation.

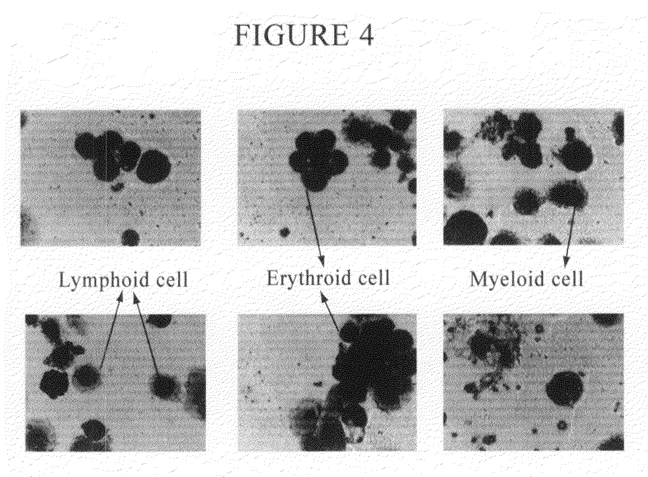

Cells were further differentiated for three weeks in serum-free methylcellulose-based medium and monitored for the formation of colonies. At the end of the clonogenic period, colonies formed were scored, harvested and subjected to cytospin separation. They were stained with May-Grunwald-Giemsa for hematologic characterization of eritroid, limphoid and myeloid subpopulations as illustrated in FIG. 4.

TABLE

List of primers used for gene expression analysis

| | EMBL | | | |
|---|---|---|---|---|
| | Accession | | Annealing | Fragment |
| GENE | No. | Primer sequence | temperature | size |
| Oct-4 | XR_000266 | Forward | 55° C. | 126 bp |
| | | 5′-acatcaaagtcttgcagaaagaact-3′ | | |
| | | Reverse | | |
| | | 5′-ctgaataccttcccaaatagaaccc-3′ | | |
| Nanog | NM_024865 | Forward | 55° C. | 181 bp |
| | | 5′-cagctgtgtgtactcaatgatagattt- | | |
| | | 3′ | | |
| | | Reverse | | |
| | | 5′-acaccattgctattcttcggccagttg- | | |
| | | 3′ | | |
| REX-1 | NM_174900 | Forward | 55° C. | 306 bp |
| | | 5′-cagatcctaaacagctcgcagaat-3′ | | |
| | | Reverse | | |
| | | 5′-gcgtacgcaaattaaagtccaga-3′ | | |
| IFN-τ | NM_176891 | Forward | 57° C. | 192 bp |
| | | 5′-gaggaaaaccacacggagaa-3′ | | |
| | | Reverse | | |
| | | 5′-aggcacaggtgctgtagtcc-3′ | | |
| BMP-4 | D30751 | Forward | 57° C. | 302 bp |
| | | 5′-tcgttacctcaagggagtgg-3′ | | |
| | | Reverse | | |
| | | 5′-aatggcatggttggttgagt-3′ | | |
| NF-H | NM_021076 | Forward | 57° C. | 250 bp |
| | | 5′-agagctggaggcactgaaaa-3′ | | |
| | | Reverse | | |
| | | 5′-tccgacactcttcaccttcc-3′ | | |
| α- | NM_004038 | Forward | 45° C. | 476 bp |
| amilase | | 5′-aattgatctgggtggtgagc-3′ | | |
| | | Reverse | | |
| | | 5′-cttatttggcgccagcgatg-3′ | | |

### BIBLIOGRAPHY

1. Kim N W, Piatyszek M A, Prowse K R, Harley C B, West M D, Ho P L, Coviello G M, Wright W E, Weinrich S L, Shay J W. Specific association of human telomerase activity with immortal cells and cancer. Science. 1994 Dec. 23;266(5193):2011-5.
2. Armstrong L, Lako M, Lincoln J, Cairns P M, Hole N. mTert expression correlates with telomerase activity during the differentiation of murine embryonic stem cells. Mech Dev. October 2000;97(1-2):109-16
3. Amit M, Carpenter M K, Inokuma M S, Chiu C P, Harris C P, Waknitz M A, Itskovitz-Eldor J, Thomson J A. Clonally derived human embryonic stem cell lines maintain pluripotency and proliferative potential for prolonged periods of culture. Dev Biol. 2000 Nov. 15;227(2):271-8.

1. A method in vitro for establishing pluripotent cell lines from human parthenotes, which comprises:
a) exposing a non-inseminated human oocyte to ionomycin and 6-dimethylaminopurine (6-DMAP), to induce its parthenogenetic activation;
b) maintaining the activated oocyte in culture conditions permissive for its development to blastocyst;
c) isolating the inner cell mass (ICM) and plating the ICM cells on a STO fibroblasts feeder layer, whereby cell colonies are formed;
d) separately propagating the cell colonies on STO-fibroblast feeder layers, whereby cell lines are established;
e) selecting the cell lines expressing, as human stem cell markers, Oct-4, Nanog, Rex-1, alkaline phosphatase, SSEA-4, TRA 1-81.

2. A method according to claim 1, wherein the oocyte activation in step a) is carried out by exposing the cells to 5 μM ionomycin followed by incubation with 2 mM 6-DMAP at 37° C.

3. A method according to claim 1, wherein step c) comprises i) removing the zonae pellucidae by incubating the blastocyst in the presence of pronase and ii) plating the ICM cells on STO fibroblast feeder layers freshly inactivated with mitomycin-C.

4. A method according to claim 1, wherein in step d) the cell colonies are propagated on STO feeder layer in low glucose DMEM/F10 (1:1) medium supplemented with LIF and βFGF.

5. A pluripotent human parthenogenetic stem cell obtainable by the method of claims 1-4, said cell expressing the following markers: Oct-4, Nanog, Rex-1, alkaline phosphatase, SSEA-4, TRA 1-81.

6. A cell culture containing the pluripotent human parthenogenetic stem cell of claim 5 and a medium suitable for its survival or propagation.

7. A method in vitro for producing a differentiated cell or tissue which comprises:
providing a culture of pluripotent parthenogenetic human stem cell according to claim 5;
exposing said culture to conditions or stimuli permissive for pluripotent parthenogenetic human stem cell differentiation.

8. A method according to claim 6, wherein said differentiated cell is selected from neuron, muscle cell, cardiomyocyte, adipocyte, epithelial, liver, cartilage or hematopoietic cell.

9. A method of treating a diseases in a human subject, which comprises administering to, or transplanting into said subject a pluripotent human parthenogenetic stem cell according to claim 5.

10. A method according to claim 9, wherein the disease is selected from neurological and neurodegenerative disorders, particularly Parkinson's and Alzheimer's diseases, spinal cord injuries, muscular distrophy, heart diseases, graft-vs-host disease, diabetes, multiple sclerosis, vascular diseases, hypercholesterolemia, age-related disturbances, ulcers, burns, rejection of transplanted organs or tissues.

11. A method for delivering therapeutic genes to individuals affected by genetic disorders, which comprises:
transfecting a pluripotent human parthenogenetic stem cell according to claim 5 with a therapeutic gene;
administering or transplanting into said individual the genetically-modified pluripotent human parthenogenetic stem cell.

12. A method according to claim 11, wherein the genetic disorder is selected from cystic fibrosis, breast cancer, hemophilia, Huntington's disease, muscular dystrophy, schizophrenia, sickle cell anemia, spine bifida.
